(19) United States
(12) Patent Application Publication
(10) Pub. No.: US 2016/0287207 A1
(43) Pub. Date: Oct. 6, 2016

(54) SMART MEDICAL EXAMINATION AND COMMUNICATION APPARATUS

(71) Applicant: Yan Xue, Fremont, CA (US)

(72) Inventor: Yan Xue, Fremont, CA (US)

(21) Appl. No.: 14/676,930

(22) Filed: Apr. 2, 2015

(51) Int. Cl. A61B 7/04; A61B 5/024; A61B 5/00; A61B 5/08; A61B 5/0402; A61B 5/145; A61B 5/01; A61B 5/026

(52) U.S. Cl. CPC A61B 7/04; A61B 5/01; A61B 5/024; A61B 5/026; A61B 5/0816; A61B 5/0402; A61B 5/14542; A61B 5/14532; A61B 5/0022; A61B 5/0006; A61B 5/0008; A61B 5/6823; A61B 5/7203; A61B 5/7271

(57) ABSTRACT

An embodiment of a portable sensor input device operatively connects to a smartphone, wearable computer device, tablet PC, and/or other mobile computer device to facilitate medical examination and diagnosis. The sensor input device used for biological signal detection contains one or more sensors inside it's chest-piece, such as, without limitation, acoustic sensor, temperature sensor, pressure sensor, light sensor, image sensor, electrocardiogram (ECG) sensor, and/or ultrasonic sensor. The ergonomically designed and shaped input device can employ an energy harvesting module to power up the sensor system. A medical application software inside the mobile computer device controls the sensor input device and performs the major functions.

### TECHNICAL FIELD

The present invention relates to one or more methods, apparatuses, and/or systems for biosignal/data detection and processing, medical measurement and diagnosis, and communication. More specifically, a portable sensor input device operatively connects to a mobile computer to facilitate medical examination and diagnosis.

### BACKGROUND

Auscultation is a primary and frequently used technique for medical examination, testing, and diagnosis. Auscultation technique requires professional medical training and substantial clinical experiences. Traditional acoustic stethoscope provides poor sound qualities and has no recording, analysis or communicating capabilities. Many doctors and other medical professionals often extend the stethoscope tube length for comfortableness, which causes more noises. Current digital stethoscopes try to solve the problem by employing many electronic components, digital processor, memory, battery, LED screen, and other hardwares. They are heavy, fragile, power-hungry and expensive. Therefor, majority of medical professionals are still using the traditional acoustic stethoscopes that were invented more than one hundred years ago.

Doctors, nurses, and medical workers are using stethoscopes everyday, but current stethoscopes, including digital stethoscopes, have very limited functions and offer few medical analysis, diagnosis and communication functions like checking cardiac arrhythmia, analysing heart murmurs, detecting crackles and wheezing sounds from lung, and communicate the result to remote locations. Separate special-purpose computer systems offer those functions, but they are expensive, the hardware and software setup are complicated, and requires professional skills and trainings.

Currently, applications like at-home telemedicine and virtual medical assistance are still very limited because there is no easy way to do at-home multiple medical measurements, and it is hard to communicate the results to remotely-located medical professionals. The measurement of a patient's condition requires many different devices and machines. It's cumbersome and in a lot of cases, not feasible for a patient to have all those medical devices and machines at home, let alone the daunting tasks of operating the devices, collecting, recording, organizing, and communicating the results. Without a reliable and multifunctional medical examination and communication system, medical professionals can not advise patients and provide diagnosis and treatments remotely.

Current general-purpose mobile computers like smartphones and tablet PCs already have the hardware and software resource to provide superb data processing and communication services, and can be utilized for sensor data processing, analysis and communication. Their audio signal processing hardware and software systems can be customized to offer high quality signal processing, filtering, and enhancement of low-frequency auscultation sound. The presented invention utilizes the hardware, software, and even power resource of general-purpose mobile computers to eliminate the need for expensive, fragile and cumbersome components employed by current digital stethoscopes and other medical devices, and minimize the need for special-purpose computer systems. Embodiments without tubes and ear pieces can improve auscultation sound quality. Embodiments also provide better user interface and communication means. By integrating multiple medical sensors for various medical examination and test purposes, the invention simplifies the medical examination process, and make it easy to record, organize and communicate the results and related information.

### SUMMARY

The present invention is about a portable sensor input device operatively connected to a mobile computer device via the audio jack to make a versatile medical device for auscultation, biosignal data collection and measurement, medical analysis, diagnosis, and communication. At least one acoustic sensor is placed in the chest-piece housing of the sensor input device to detect sound from inner organs and transmit the acoustic signals to a mobile computer device for further sound processing, enhancement, and analysis. The sensor input device may also integrate one or more additional sensors, such as, without limitation, temperature sensor, electrocardiogram (ECG) sensor, pressure sensor, light sensor, image sensor, and/or ultrasonic sensor. The sensor input device comprises an ergonomically designed chest-piece with flexible gooseneck joint to fit users' hand and body positions for comfortableness and single hand operation. An energy harvesting system converting audio, light, and/or heat energy wave into electric current can be used to power up the sensor system inside the chest-piece. Embodiments utilizing the hardware and software resources of a mobile computer device to perform major functions and/or power up the sensor system can minimize the need of electronic components and parts that have been used on a digital stethoscope or other medical examination devices, such as, without limitation, data processor, digital memory, hardware storage, LED screen, and/or battery.

An embodiment of a mobile-computer-empowered medical examination and communication apparatus includes a medical software application inside the mobile computer to control the sensor input device and perform major functions, like signal processing and enhancement, data measurement and analysis, medical diagnosis, multimedia user interface, and real-time communication, as but a few examples. Some of the function features and methods of the claimed subject matter including, without limitation, multimedia display, speech recognition, text-to-speech, voice memo, messaging, information sharing, and real-time audio/video chat are not previously associated with stethoscopes or other medical examination devices. The present invention can bring biosignal detection, data processing and enhancement, medical measurement, diagnosis, and communication functions all together in one place, so that physicians and medical professionals can easily collect and track patient information, get instant medical analysis, and provide real-time medical assistance and/or treatments.

The invention comprises a sensor input device, physical embodiment, detecting biological signals and a method of connecting to a mobile computer device to perform major functions. The claimed subject matter is not limited in this regard, any modifications, adaptations, or variations that rely upon the method of the present invention are considered to be within the spirit and scope of the present invention. For example, the physical design and appearance of the sensor input device may vary as the shapes, colors, size and materials can be customized for different uses. In some situations, the sensor input devices may utilize Bluetooth®, WiFi, ZigBee, and/or other wireless technology to operatively connect with the mobile computer, instead of using the audio jack or data dock. Wired and/or wireless network transmissions can be used to connect multiple sensor devices and/or medical equipments in various locations with mixed settings where alternate sources of potential signal interference can be present. A mobile computer device includes, without limitation, smartphones, tablet computers, wearable computers, PDAs and/or other handheld devices, and other computer devices and/or electronic devices.

### BRIEF DESCRIPTION OF THE DRAWINGS

FIG. 1 illustrates an embodiment of a sensor input device operatively connected to a smartphone via the audio jack to make a medical examination and communication apparatus consistent with the claimed subject matter.

FIG. 2 shows a general view of an input device.

FIG. 3 illustrates a sample components and parts of an input device with integration of sensors in the chest-piece.

FIG. 4 illustrates the major function aspects of presented invention.

FIG. 5 is a function block diagram of the claimed subject matter.

FIG. 6 shows the data access and digital communication system.

### DETAILED DESCRIPTION

In one embodiment, presented herein for illustrative purposes and without limitation, the claimed subject matter comprises a sensor input device operatively connected to a smart phone, tablet PC, or wearable computer device to make a versatile medical apparatus for auscultation, medical examination and measurement, diagnosis, and communication functions. In one embodiment, for example, the presented invention can be used as an electronic stethoscope to detect, monitor, and/or record acoustic sounds from inner organs such as hearts, lungs, or intestines. Embodiments can also be constructed to support other types of medical examination functions by integrating one or more additional sensors including, without limitation, temperature sensor, electrocardiogram (ECG) sensor, pressure sensor, light sensor, image sensor, and/or ultrasonic sensor. An energy harvesting system converting audio, light, and/or heat energy wave into electric current can be used to power up the sensor system, without the presence of a battery inside the sensor input device. The energy harvesting system may take the audio output of the mobile computer as energy conversion source, or absorb natural light and/or heat wave as energy source to power up the system. An embodiment of utilizing the hardware and software resources of a mobile computer device to control the input device and perform major functions can minimize the need of electronic components and parts that have been used on a digital stethoscope or other medical examination devices, such as, without limitation, data processor, digital memory, hardware storage, and LED screen. Sensor components can be easily removed, replaced, and/or added on the sensor input device because of such simplified electric circuit system inside the sensor input device. The combination and contains of the sensor components, including the acoustic sensor for auscultation purpose, may differ based on the applications or uses.

Connecting the medical sensor input devices with mobile computers can also bring biological signals detection, data processing and enhancement, medical measurement, monitoring, diagnosis, and communication functions all together in one place, so that physicians and other professionals can easily collect and track patient information, get instant medical analysis, and provide real-time medical assistance and/or treatments. The presented invention can provide medical examination functions like, auscultation, measuring body temperature, testing blood pressure or glucose level, taking ultrasound or ECG remotely at home or on the road. Authorized doctors or medical professionals can control and/or adjust setting and preferences of the sensor input device via the Internet to virtually assist or guide a patient taking examinations or monitoring medical conditions. Auscultation audios, sensor signal graphs, medical data and record, and other user information can be saved on the local mobile computer device and/or on a cloud computer. Mobile computer devices include, without limitation, smartphones, tablet computers, wearable computer devices, PDAs and/or other handheld devices, laptop, palmtop, and/or other computer devices and/or electronic devices. The claimed subject matter, however, is not limited to the illustrative characteristics described with respect to these embodiments. Embodiments can employ additional or alternative computer devices, standards, and/or technologies, now known or later developed, consistent with the claimed subject matter.

In one embodiment, an ergonomically designed and shaped sensor input device comprises a chest-piece with a flexible gooseneck joint connecting to the mobile computer device via the audio jack or data dock for comfortable single hand operations. The flexible gooseneck joint is strong enough to hold the chest piece in place while it can also be easily twisted or bent in different directions to fit a user's hand and body positions. For example, the audio jack plug at the end of the flexible gooseneck joint linking the sensor system with a smartphone or tablet PC can transmit sensor signals and/or digital communication data through the standard 3.5 mm audio port. User can plug in the sensor input device to a smartphone for examinations or check-ups, and simply unplug it if they don't use the device. In some implementations, wireless data transmission and communications between the sensor input device and mobile computer may be used for hygienic reasons, cross infection prevention, or personal preferences. In such cases, the sensor input device can contain a wireless transmitter utilizing the Bluetooth, WiFi, Zigbee, and/or other wireless technology to offer compatibility with various wireless protocols and standards. The claimed subject matter is not limited in this regard, and any modifications, adaptations, or variations that rely upon the method of the present invention are considered to be within the spirit and scope of the present invention.

Embodiments comprise a medical software application running on the mobile computer devices to control the sensor input device and process the raw signals derived from the sensors inside the chest-piece, such as, without limitation, amplifying and filtering signals, converting data from analog-to-digital and/or digital-to-analog, data sampling, and/or noise canceling. The software application also supports medical measurement means by calculating and analyzing heart or respiration rate, body temperature, blood pressure, blood oxygen level, or glucose level, as but a few examples. Those medical measurement results and information can be further analyzed and compared with medical standards and references to provide diagnostic suggestions or advices to patients. The software application comprising medical decision logic functions can automatically detect warning signs and/or assist patients with corresponding treatment or resolutions based on the diagnostic results. The enhanced sensor data, medical measurement and diagnosis results, among with other patient information can be updated to a cloud computer in real-time and/or shared with one or more authorized users or professionals instantly via email, messaging, social media, audio/video chat, and/or other outside communication platforms over the Internet.

The software application supports user interface means including multimedia display, audio input/output, user information collection and storage, user preference and function selections. The mobile computer device provides hardware and software system support for the user interface and communication functions, and the software application modifies and enhances the system specifically for medical examination, diagnosis, and communication purposes. For example, filtered, amplified and enhanced auscultation sound can be played on the mobile computer device and/or audio output systems via the wired and/or wireless network system. Users can view the sensor signal graphs, measurement results, medical references and records, and/or messages on the screen of the mobile computer device and/or a connected output display screen. Speech recognition systems can be utilized for converting voice input into text, text-to-speech, data-to-speech functions to provide a user friendly communication environment, which is especially important for older or disabled users. Authorized doctors and/or medical professionals can remotely access the signal or data outputs, examination and diagnosis results, medical records, and other patient information in real-time via the Internet.

For illustrative purposes and for facilitating disclosure, reference will be made herein to the embodiments illustrated in the drawing figures. It should be noted however, that these embodiments and their respective component embodiments are presented for illustrative purposes only, and not by way of limitation. Additional and/or alternative embodiments and/or components can also be employed consistent with the claimed subject matter.

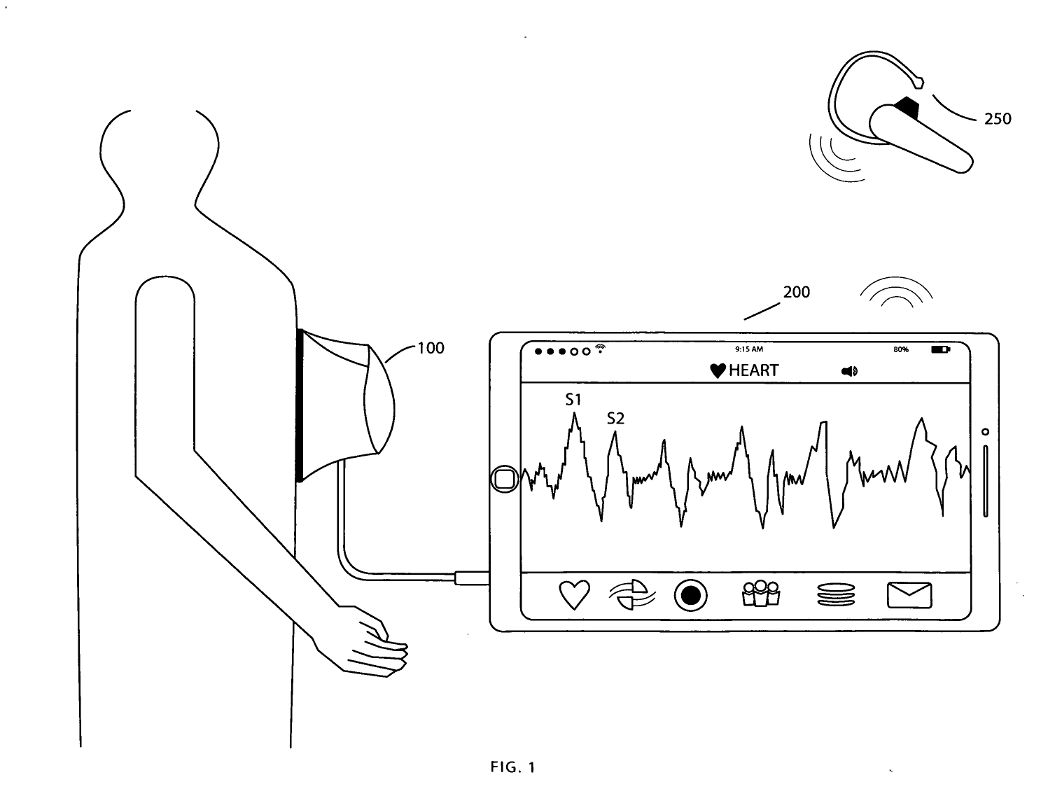

Presented invention contains both a sensor input device, physical embodiment, and a method of utilizing the computing, network, and/or power resource of a mobile computer device to control the sensor input device and perform the major functions. In one embodiment, for example, FIG. 1 shows a sensor input device 100 operatively connecting to a smartphone 200 via the audio jack for auscultation, examination, and communication purpose. At least one acoustic sensor, microphone, is embedded in the chest-piece housing to detect the sound signals from inner organs. Embodiments support different operating modes, different frequency emphasis, such as 20-200 Hz bell mode for heart auscultation, 100-500 Hz diaphragm mode for lung examination, and/or 20-1000 Hz combined mode for general purposes. Acoustic signal graphs and heart/respiration rates can be view on the screen of the smartphone. Auscultation sound can be played on the audio output system of the smartphone and/or via the Bluetooth earphones 250.

The medical software application amplifying, filtering, and enhancing the raw signals derived from the acoustic sensor to improve the auscultation sound quality may help doctors and other medical professionals to detect abnormal heart murmurs, crackle and wheezing sounds easily, and aid users with hearing difficulties. Embodiments drawing graphical sensor signals to reflect the physical movements of inner organs on the smartphone's screen can provide an intuitive way to understand and analyze the sensor data. Current digital stethoscopes do not have big LED screens to show the graphical signals directly on the device, and offer insufficient data analysis and graphic function. The presented invention providing visualized data analysis and historical measurement data graph can facilitate early diagnosis and empower user with augmented intelligence. The input sensor device, physical embodiments, offering flexible compatibility in the type and/or style of the computers and devices connected can make the medical examination and communication apparatus more portable, convenient, and economical for users.

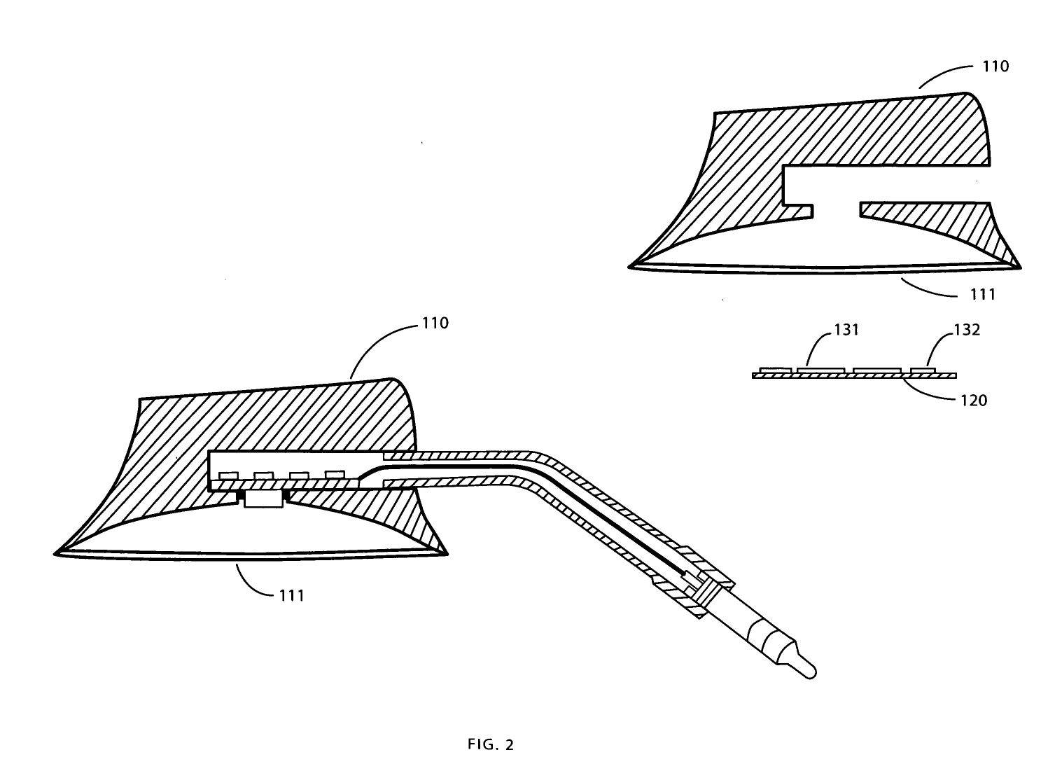

For illustrative purposes, and not by way of limitation, FIG. 2 shows a general view of the sensor input device that can be sized, shaped, formed, and/or constructed so as to be easily and/or comfortably held and operated using one hand and placed against a body during use. The chest-piece 110 detecting biological signals from a living body comprises one or more sensors, such as, without limitation, acoustic sensor, temperature sensor, oximetry sensor, pressure sensor, image sensor, ultrasonic sensor, and/or motion sensor. The flexible joint 120, made by metal gooseneck or other flexible arm, is hard enough to hold the device in place, but also can be bended to any direction to fit user's gesture. At one end of the flexible joint, an audio jack plug 130 connects the sensor system with a mobile computer device. Raw signals and/or data derived from the sensors inside the chest-piece can be transmitted to the mobile computer device via the audio port. Major data processing and enhancement functions including, without limitation, amplification, filtering, data sampling, analog-to-digital and/or digital-to-analog conversion, and noise canceling are performed by the medical software application utilizing the hardware and software resources of the mobile computer device.

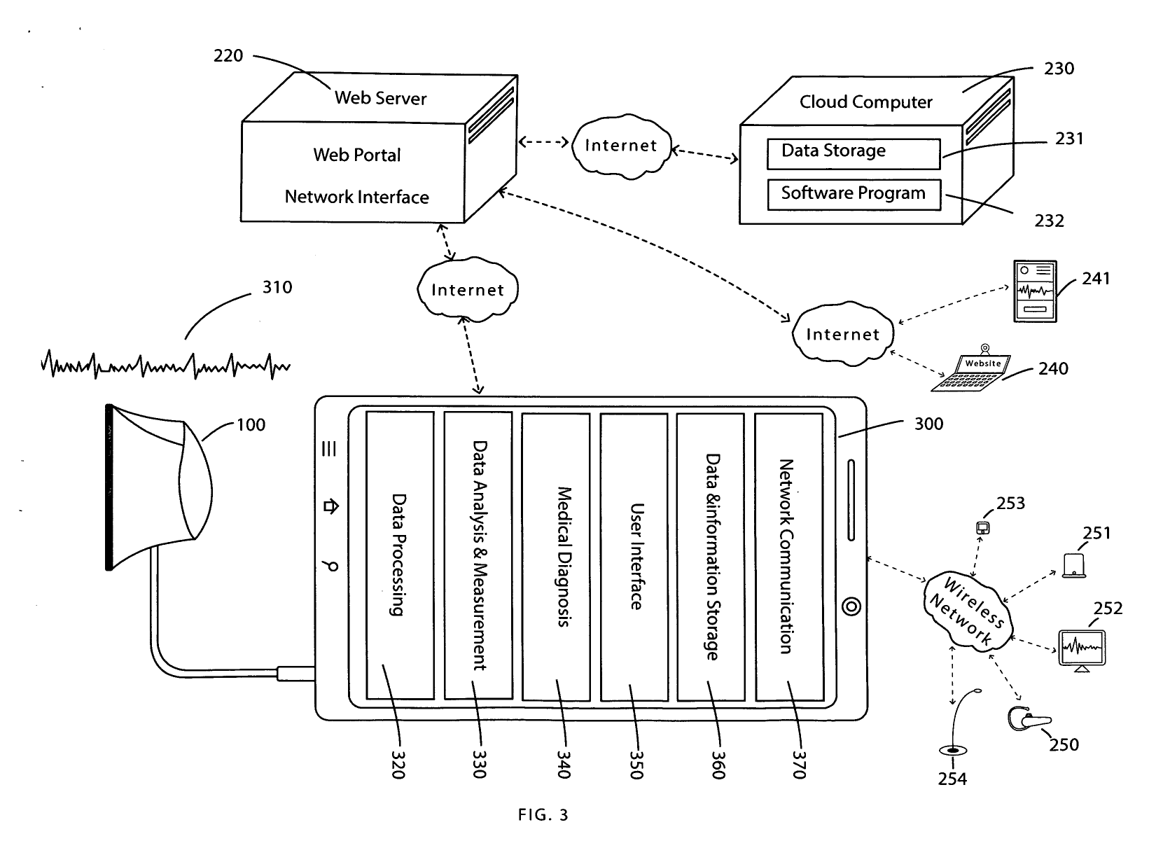

As an example, FIG. 3 shows a few electronic components and parts that can be used to assemble a sensor input device with the claimed subject matter. With detailed reference to FIG. 3, examples of electronic components and parts may include a chest-piece housing 110, a diaphragm 111, a printed circuit board (PCB) 150, energy harvesting components 151, acoustic sensor 152, other sensors 153, a flexible gooseneck joint 120, and an audio plug 130. The sensor input device contains an acoustic sensor 152 placed close to the diaphragm for better sound detection. Embodiments may also integrate one or more additional sensors inside the chest-piece to detect different kinds of biological signals, such as, without limitation, temperature sensor, oximetry sensor, pressure sensor, image sensor, ultrasonic sensor, and/or motion sensor. The sensor input device containing an energy harvesting component 151 on the PCB board to convert audio, light, and/or heat energy into electric current to power up the sensor system without the presence of a battery or electric power charging sources. Electric wires 121 inside the flexible gooseneck tube connecting the sensor system with the mobile computer device can transmit raw sensor signals. Of course, those skilled in the relevant art will appreciate that fewer, additional, and/or alternative electronic component embodiments could be used, depending at least in part on the desired implementation, while still be consistent with the claimed subject matter.

In one embodiment, a digital stethoscope like electronic auscultation device comprising one acoustic sensor, an electret condenser microphone, inside the chest-piece housing to detect acoustic signals from inner organs acting as a passive component of the connected mobile computer device may not need any power source to operate. However, if additional and/or alternative electronic sensors and components are integrated, embodiments may employ an alternative energy harvesting component transforming audio, light, radio, heat, and/or mechanical energy into electric energy. For an example, the energy harvesting component can effectively convert the audio wave energy sending out from the mobile computer's standard 3.5 mm audio output port into electric energy to power up the electronic sensors and components. In some implementations, a solar energy, heat energy, or radio frequency energy harvesting method may be preferred to minimized the energy consumption of the mobile computer device. Utilizing those alternative energy sources to power up the sensor system can eliminate the need of a battery and/or electric power charging components for the sensor input device. A sensor input device with no battery and/or electric power charging components required can be smaller, lighter, and more appealing to users.

In some implementations, a wireless sensor input device utilizing Bluetooth, WiFi, ZigBee, and/or other wireless technology to operatively connect with a computer device may be desired for data transmission and/or information exchange. In such cases, the circuit design, shape, and appearance of the sensor input device can vary to adapt the functional changes. For example, a sensor input device, the physical embodiments, may integrate additional and/or alternative electronic components like wireless transceiver, microcontroller, and/or antenna inside the chest-piece to facilitate the wireless communication function between the sensor input device and a computer device. In addition, components and parts like flexible joint and audio plugs can be removed or detached from the sensor input device to adapt the changes. A wireless sensor input device compatible with all kinds of computing devices, including, without limitation, mobile computer devices, personal PCs, and/or laptop PCs can increase the portability and extend usefulness of the sensor input device, but require custom circuits and consume more energy. The wireless sensor input device can offer compatibility with various wireless and network protocols or standards, and any variation, adaptation, or modifications that rely upon the method of the present invention are considered to be within the spirit and scope of the present invention.

For illustration purpose, the sensor input device showed on FIG. 3 only contains an acoustic sensor. However, additional and/or alternative sensors can be inserted or detached, such that they become accessories to be added to the sensor input device. In such a case, the chest-piece housing 112 of the sensor input device can be designed to contain spaces for inserting the various sensor and electronic components and parts. Therefore, the presented invention can have broader application in medical examination and measurement field, such as, without limitation, measuring body temperature or blood pressures, testing blood oxygen or glucose level, taking electrocardiography (ECG), and/or taking ultrasound images. The presence of acoustic sensor inside the chest-piece housing is not required in some implementations where the auscultation function is unnecessary. The claimed subject matter is not limited in this regard, any modifications, adaptations, or variations considered to be within the spirit and scope of the present invention.

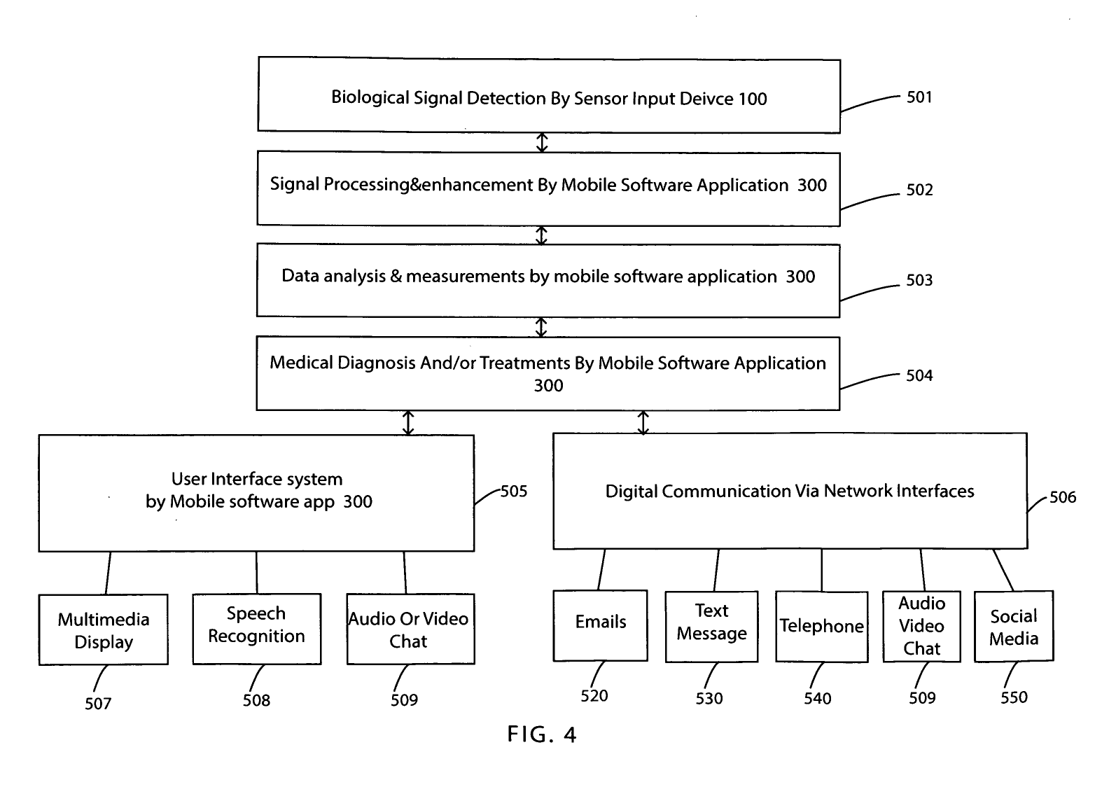

FIG. 4 illustrates the major function aspects of presented invention including, without limitation, sensor signal and/or data collection 310, data processing and enhancement 320, medical analysis and measurement 330, user input system 350, multimedia output and display 340, information and/or data storage 360, and real-time network communications 370. In one embodiment, the sensor input device employing multiple electronic sensors to detect various biological signals for different medical measurements supports a versatile healthcare and communication platform which can aggregate and simplify the examination medical processes. For example, embodiments measuring and presenting a patient's body temperature, heart rate, blood pressure, blood oxygen level, and auscultation signal graph promptly can give the doctor a comprehensive view of the patient's medical or health condition, and may also lead to a clue for possible diagnosis or treatment. The presented invention also provides better user interface experiences that digital stethoscopes or other medical devices are missing, such as, without limitation, digital multimedia outputs and display 507, voice input or command, speech-to-text or data/text-to-speech 508, audio or video chat 509, and/or messaging. Embodiment comprising multimedia outputs and display system may employ alternative and/or additional components, hardware, and/or devices, such as, without limitation, a Bluetooth earphone 250, a large display monitor 252, or a wireless speakers 251. Those user interface functions and methods can improve the application and usage of telemedicine system. The presented invention offering broad compatibility with commercially available mobile computer devices can improve mobility and expedite the process of many medical examination and diagnostic procedures like, auscultation, patient health monitoring, ECG, and/or ultrasound, as but a few examples.

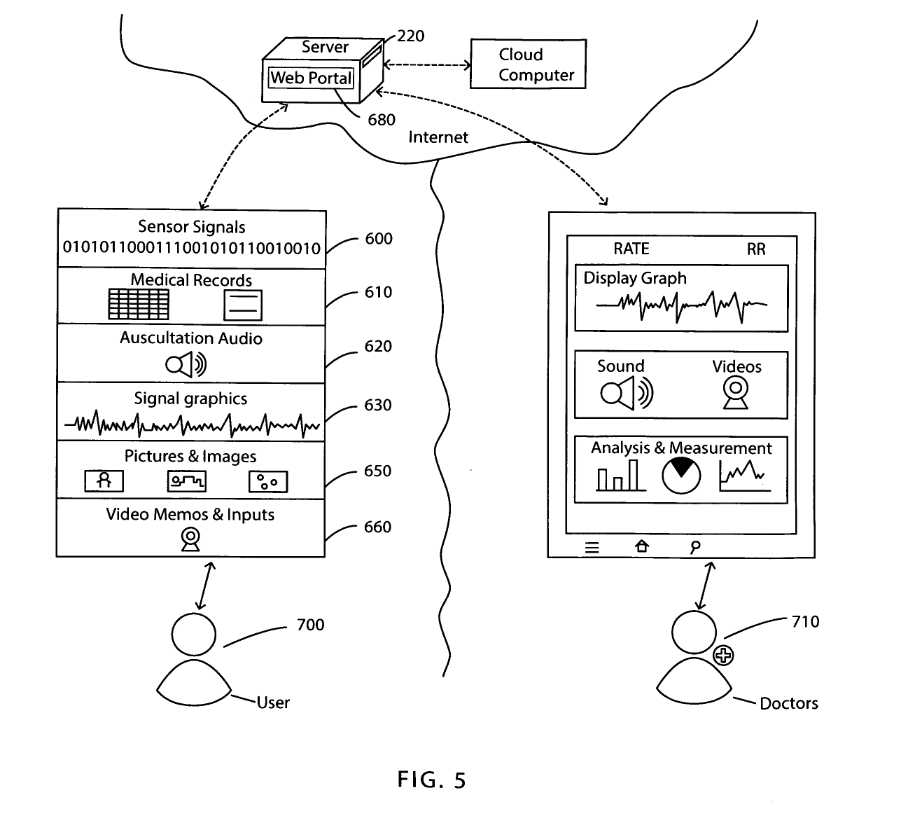

For additional illustration purposes, FIG. 5 is a function block diagram of the claimed subject matter. A sensor input device detects biological signals 501 from a living body to support various medical examination functions. The connected mobile computer device 200 provides hardware, firmware, software, and/or digital storage for the apparatus to process and enhance the data derived from the sensors inside the input device. A medical software application 300 inside the mobile computer device control the sensor input device and performs major functions including, signal processing and enhancement 502, data analysis and measurements 503, medical diagnosis and/or treatments 504, and communication functions 506. Data and information like sensor data, auscultation audios, user inputs, medical records, graph, videos, and/or images can be stored in the mobile computer device's digital storage space and/or on the cloud computer 230 via the Internet. Authorized users like doctors and other medical professionals can access the user data and information remotely using their own computers 240 or smartphones 241 via the secured web server 220. The web server can provide user authentication and data encryption functions to secure the data access and communication. Embodiments utilizing Software Application As Service (saas) programs 232 on the cloud computers to analyze and study sensor data may offer augmented intelligence in medicine. The medical software application utilizing multimedia, voice recognition, and digital data communication technologies to support better interface means 505 may employ additional and/or alternative devices, components, or machines, such as, without limitation, Bluetooth earphones 250, wireless speakers 251, large screen displays 252, video cameras 253, and/or teleconference systems 254.

For example, an auscultation process starts from placing the sensor input device against a living body to detect sound signals from the inner organs. As a peripheral, the sensor input device does minimal data processing work by directly sending the raw signals derived from the sensors to a smartphone for further data process, enhancement, and analysis. The medical software application inside the smartphone can filter out noises and unwanted sound, amplify and emphasize low frequency auscultation sound, convert the analog sound signal into digital data and/or convert digital data into analog sound, draw graphs of the processed signals, as but a few examples. The medical software application can emphasize on a special frequency range for each different operation mode, such as, 20-200 Hz for Bell mode, 100-500 Hz for Diaphragm mode, and/or 20-1,000 Hz for Extended Range mode. The graphical auscultation signal wave can be viewed on the screen of a smartphone and/or other connected computers or devices via a wired or wireless network, and the audio record can be played via the smartphone and/or bluetooth speakers. Those auscultation signal graph and audio files along with other medical information and records can be transmitted to a cloud computer, where doctors and/or other authorized users can access in a real-time fashion. Embodiments can also directly transmit the sensor data and other medical information to doctors and other medical professionals via email, messaging, social media, and/or other communication system or platforms.

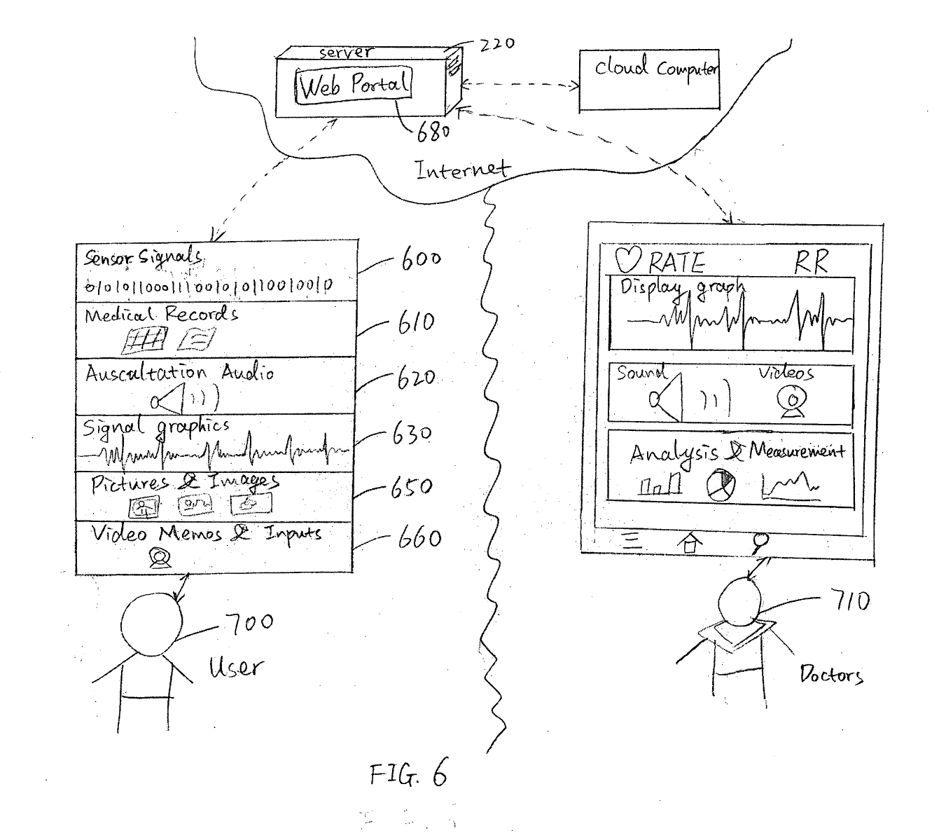

FIG. 6 shows the data access and digital communication system of the presented invention. Embodiments utilizing the network hardware, firmware, and software systems of the mobile computer device to provide digital communication functions may employ some networks and technologies like machine-to-machine data transmission, near field communication, radio communication, voice over IP, telecommunication, and sensor network communication, as but a few examples. Embodiments can offer broad compatibility with various network and communication software and protocols. In one embodiment, the medical software application provides data and information sharing or exchange functions via electronic mail 520, text messaging 530, telephone system 540, audio or video chat 509, social media 550, and/or other communication platforms or systems. The invention comprising communication means supports a wide range of data types including, but not restricted to, analog signals or digital data derived from the sensors 600, medical information and record 610, auscultation audio 620, sensor signal graphics 630, pictures and/or images 650, and/or voice memos or inputs 660.

In some implementations, embodiments can comprise a wireless sensor input device which offers compatibility with various wireless protocols, such as, without limitation, IEEE 802.11, Bluetooth Classic, Bluetooth Smart, ZigBee, and/or later specification standards. The wireless sensor input device transmits raw sensor signals to the mobile computer device via wireless network, and the medical software application inside the mobile computer device can wirelessly control and/or configure the wireless sensor device. The data processing and enhancement, medical measurement, diagnosis, communication with outside networks and systems, and other related functions performed by the medical software application are exactly the same for embodiments with wireless sensor input devices.

Embodiments encompassing health monitoring and alert functions can automatically notify patients or doctors if the medical software application detects any abnormal medical examination results, suspected sensor signals and/or symptoms, or other pre-defined medical conditions or criterias. The notification message can be voice and/or graphic alert on the mobile computer device, and/or it can be sent to the doctors via email, text messaging, and/or automatic telephone system, as but a few examples. A web portal 680 comprising authentication and encryption means can authorize the doctors to access a patient's real-time sensor data, measurements, analysis, and other medical information in a secure and safe cyber environment. The web portal serving as a communication hub bridges and/or facilitates the medical examination and communication apparatus with the outside computer and device systems or platforms to facilitate real-time communication and telemedicine functions including, without limitation, data backup and update on the cloud computer, virtual medical assistance, remote medical diagnosis or consulting, and/or health monitoring.

For example, with the user's 700 consent, authorized physicians or other medical professionals 710 can access a patient's examination data and/or medical information remotely using their computers and devices via the web portal. In some implementations, a doctor or other medical service provider may remotely change or adjust the preference setting of the medical software application inside a mobile computer device to assist the user in controlling the sensor input device, choosing operating mode, and/or tracking or monitoring medical symptoms, as but a few examples. Embodiments comprising digital communication means can also facilitate multi-party teleconference via the Internet where numerous types of devices and/or equipments in various locations with mixed settings may be present. The web portal create and manage the teleconference sessions, and audio/video conference server may be utilized to facilitates the data transactions during the conference. The teleconference functions are especially useful to conduct telemedicine or medical education and research.

An embodiment of a medical examination and communication apparatus empowering patients with augmented artificial intelligence and mobility can provide instant medical help, improve self health awareness and monitoring, and/or minimize physical clinic visits. Embodiments can be smarter, more interactive and intuitive as mobile computing and/or cloud computing technology advances. The medical software application comprising signal/data processing, analysis, and diagnosis functions can utilize the inside computer processing power, hardware storage, and/or memory space of a local mobile computer device to perform the major functions, and/or transmit the enhanced sensor data and related medical information to a cloud computer for further analysis, diagnosis, and/or storage. Centralized medical data processing centers located on cloud computers can automate complex data analysis, medical measurement, and/or diagnosis tactics, such as, without limitation, analyzing heart murmurs, detecting one or more frequency ranges corresponding to pathologies associated with particular diseases, detecting crackles and wheezing sounds from lung, reading electrocardiographic (ECG), and/or diagnostic ultrasound measurement. Many advanced data processing and/or analyzing technology can be utilized by the cloud computer to provide automatic medical diagnosis, virtual medical assistance, and/or treatment.

The claimed subject matter is not limited in this regard, any modifications, adaptations, or variations that rely upon the method of the present invention are considered to be within the spirit and scope of the present invention. For example, the physical design and appearance of the electronic peripheral device may vary as the shapes, colors, size and materials can be customized for different users. The chest-piece may integrate one or more sensors such as acoustic sensor, temperature sensor, pressure sensor, light sensor, image sensor, electrode, ultrasonic sensor, chemical sensor, an electrical activity sensor, a far infrared sensor, a near infrared sensor, and/or other electronic components. In some implementations, embodiments may be used in various situations with mixed settings where numerous types of equipment or alternate sources of potential signal interference can be present. For example, embodiments can be used by doctors as medical examination devices, or used by patients as homecare medical devices. Embodiments can also be used in hospitals and colleges for medical training purposes, or connected to a network system to support telemedicine and/or virtual medical assistance. Hence, these descriptions and drawings are not to be considered in a limiting sense, as it is understood that the present invention is in no way limited to the embodiments illustrated.

1. A portable medical examination and communication apparatus, comprising:
a.) a sensor input device operatively connecting to a mobile computer device for biological signal detection and transmission;
b.) a medical software application inside the mobile computer device to control the sensor input device and perform major functions, such as, without limitation, signal processing, data analysis, medical examination and diagnosis, real-time communication, and/or telemedicine.

2. A portable medical examination and communication apparatus according to claim 1, further including an ergonomically designed and shaped chest-piece with a flexible joint to fit user's hand/body position and support single hand operation.

3. A portable medical examination and communication apparatus according to claim 2, wherein a flexible joint, made by flexible metal tubing or other material flexible arm, contains an audio jack plug or a data dock plug at one end.

4. A portable medical examination and communication apparatus according to claim 1, further comprising at least one sensor inside the chest-piece of the sensor input device to detect and transmit biological signals.

5. A portable medical examination and communication apparatus according to claim 1, wherein the sensor input device may integrate one or more additional sensors including: acoustic sensor; temperature sensor; electrocardiogram (ECG) sensor; pressure sensor; light sensor; image sensor; ultrasonic sensor.

6. A portable medical examination and communication apparatus according to claim 1, wherein the sensor input device may contain an energy harvesting component to power up the sensor input device system.

7. A portable medical examination and communication apparatus according to claim 1, wherein a mobile computer device provides hardware, software, and storage to support signal processing, measurement, diagnosis, and communication means.

8. A portable medical examination and communication apparatus according to claim 1, wherein signal processing includes means for amplification, noise filtering, signal enhancement, frequency emphasis, and analog-to-digital or digital-to-analog data conversion.

9. A portable medical examination and communication apparatus according to claim 1, wherein data analysis means comprises one or more of the following calculations or measurements: heart rate; respiration rate; body temperature; blood pressure; ECG measurement; blood oxygen level; blood glucose level; ultrasonic measurement.

10. A portable medical examination and communication apparatus according to claim 1, wherein medical examination and diagnosis means includes detecting: cardiac dysrhythmias; high body temperature; high or low blood pressure; abnormal blood oxygen and/or glucose level; the frequency ranges corresponding to pathologies associated with particular diseases as derived from acoustic sensor signals.

11. A portable medical examination and communication apparatus according to claim 1 further containing user interface means supported by the medical software application for multimedia outputs, speech recognition, audio input, audio/video memo, data/text-to-speech, and/or graphical visual display.

12. A portable medical examination and communication apparatus according to claim 1, further comprising wireless communication means.

13. A portable medical examination and communication apparatus according to claim 1, wherein communication means comprises sensor signal/data transmission, medical information and data sharing, electronic mail, text messaging, audio or video chat, teleconference, and/or remote access.

14. A portable medical examination and communication apparatus according to claim 1, wherein the preference setting, operating mode, or health/symptom monitoring criteria within the medical software application can be adjusted and/or changed remotely.

15. A portable medical examination and communication apparatus comprising network and communication means supports real-time data access and/or information exchange via a web server where multiple authorized users or professionals can view the auscultation audios, signal graphic, data measurements, and/or other medical information at same time.

16. A portable medical examination and communication apparatus comprising network means for connecting multiple computers and/or medical devices in various locations with mixed settings via wired and/or wireless networks.
